(19) United States
(12) Patent Application Publication
(10) Pub. No.: US 2015/0051490 A1
(43) Pub. Date: Feb. 19, 2015

(54) ULTRASOUND SCANNING

(71) Applicant: SMITH & NEPHEW, INC., Memphis, TN (US)

(72) Inventors: Brian William McKinnon, Bartlett, TN (US); Kevin Weaver, Bartlett, TN (US); F. Javier de Ana, Chapel Hill, NC (US); Ruxandra Cristiana Marinescu Tanasoca, Memphis, TN (US)

(21) Appl. No.: 14/376,486

(22) PCT Filed: Feb. 6, 2013
(86) PCT No.: PCT/US13/24856

§ 371 Date: Aug. 4, 2014

Related U.S. Application Data
(60) Provisional application No. 61/596,591, filed on Feb. 8, 2012.

(51) Int. Cl. A61B 8/08; A61B 8/14; A61B 8/00

(52) U.S. Cl. CPC A61B 8/0875; A61B 8/4263; A61B 8/5207; A61B 8/523; A61B 8/4227; A61B 8/145; A61B 8/466
USPC 600/443

(57) ABSTRACT

A system for ultrasound scanning includes an ultrasound transducer assembly configured to generate ultrasound scan data indicative of surface features of bony anatomy. The system includes a deformable cuff that transmits ultrasound when filled with an ultrasound-conductive medium. The system also includes a transference mechanism configured to move the ultrasound transducer assembly relative to the deformable cuff along a path while maintaining contact between the ultrasound transducer assembly and the cuff. The transference mechanism is also configured to generate position data indicative of different positions of the ultrasound transducer assembly along the path.

### CROSS-REFERENCE TO RELATED APPLICATION

This application claims priority to and the full benefit of U.S. Provisional Application Ser. No. 61/596,591, filed Feb. 8, 2012, and titled “ULTRASOUND SCANNING,” the entire contents of which are incorporated herein by reference.

### TECHNICAL FIELD

This disclosure relates to ultrasound scanning.

### BACKGROUND

Medical imaging of bony tissue is often performed using x-ray imaging, computed tomography (CT), or magnetic resonance imaging (MRI). However, x-ray imaging techniques expose patients to ionizing radiation, and MRI equipment is generally very large and expensive. Generally, ultrasound imaging has been used as a diagnostic tool for assessing cartilage and soft tissues. Additional techniques for imaging bony tissue would be advantageous to physicians.

### SUMMARY

In a general aspect, a system for ultrasound scanning includes an ultrasound transducer assembly, a control unit, a reference, and a spatial tracking device.

In another general aspect, a system for ultrasound scanning includes an ultrasound transducer assembly configured to generate ultrasound scan data indicative of surface features of bony anatomy. The system includes a deformable cuff that transmits ultrasound when filled with an ultrasound-conductive medium. The system includes a transference mechanism configured to move the ultrasound transducer assembly relative to the deformable cuff along a path while maintaining contact between the ultrasound transducer assembly and the cuff. The transference mechanism is configured to generate position data indicative of different positions of the ultrasound transducer assembly along the path.

Implementations can include one or more of the following features. For example, the system includes one or more processors and one or more data storage devices storing instructions that, when executed by the one or more processors, cause the one or more processors to: receive the ultrasound scan data, the ultrasound scan data including data sets acquired at different positions of the ultrasound transducer assembly relative to the cuff; receive the position data, the position data being indicative of the different positions of the ultrasound transducer assembly when the data sets were acquired; and associate each of the data sets with the position data indicative of the position of the ultrasound transducer assembly when the data sets were acquired. The cuff is configured to be placed about a portion of a patient's body. The cuff has an outer surface, and the transference mechanism is configured to apply pressure between the transducer assembly and the outer surface of the cuff. The cuff defines a central opening and a central axis that extends through the opening. A distance between the transducer assembly and the central axis is variable along the path. The position data indicates positions of the transducer assembly that correspond to different distances from the central axis. The cuff has an inner surface that conforms to a portion of a patient's body placed within the cuff. The cuff includes a pressurizing mechanism. The pressurizing mechanism is a pump or a constricting element. The cuff defines a central opening and a central axis that extends through the opening, and the path includes one or more segments including motion along a plane that includes the central axis or one or more segments along a substantially helical path about the central axis.

A system for ultrasound scanning includes an ultrasound transducer assembly configured to generate ultrasound scan data indicative of surface features of bony anatomy. The system includes a reference attachable to the ultrasound transducer assembly and configured to move with the ultrasound transducer assembly when attached to the ultrasound transducer assembly. The system includes a spatial tracking device configured to communicate with the reference. The system includes one or more processors and one or more data storage devices storing instructions that, when executed by the one or more processors, cause the one or more processors to perform various operations. The operations include receiving the ultrasound scan data, which includes data sets acquired at different positions of the ultrasound transducer assembly relative to the bony anatomy from a direction substantially orthogonal to the bony anatomy. The operations include receiving position data from the reference or the spatial tracking device. The position data indicates different positions of the reference during acquisition of the ultrasound scan data while the reference is attached to the ultrasound transducer assembly. The operations include associating each data set of the ultrasound scan data with the position of the reference when the data set was acquired.

Implementations can include one or more of the following features. For example, the reference is a fiducial, and the spatial tracking device includes an optical detector. The reference is an magnetic sensor, and the spatial tracking device includes an electromagnetic field generator. The reference is a radiofrequency sensor, and the spatial tracking device includes a radiofrequency transmitter. The spatial tracking device is a mechanical linkage configured to measure movement, and the reference is an attachment element attached to the mechanical linkage. The system includes a second reference removeably coupleable to a patient's anatomy. The instructions, when executed by the one or more processors, cause the one or more processors to perform operations that include receiving second position data from the second reference or the spatial tracking device. The second position data indicates different positions of the second reference during acquisition of the ultrasound scan data while the second reference is attached to the patient's anatomy. The operations include associating each data set of the ultrasound scan data with the position of the second reference when the data set was acquired. The second reference attaches to the patient's anatomy extracutaneously with a brace, a sleeve, a band, a strap, a belt, a clip, an adhesive, or an elastic.

In another general aspect, a system includes an ultrasound transducer assembly configured to generate ultrasound scan data indicative of surface features of bony anatomy that extends along a longitudinal axis. The ultrasound transducer assembly acquires data in a scan area that has a central axis. The system includes a container configured to hold an ultrasound-conductive medium, and the container defines an opening to admit the bony anatomy. The system includes a transference mechanism configured to move the ultrasound transducer assembly within the container along one or more predetermined paths while orienting the ultrasound transducer assembly such that the central axis of the scan area is substantially orthogonal to the longitudinal axis of the bony anatomy. Each of the one or more predetermined paths includes segments in which the ultrasound transducer assembly faces the bony anatomy from different directions about the longitudinal axis. The system includes one or more processors and one or more data storage devices storing instructions that, when executed by the one or more processors, cause the one or more processors to perform various operations. The operations include receiving the ultrasound scan data. The ultrasound scan data includes data sets acquired at different positions of the ultrasound transducer assembly relative to the bony anatomy. The operations include determining a position corresponding to each of the data sets.

Implementations can include one or more of the following features. For example, the container has an inner surface and defines the one or more predetermined paths on the inner surface, and the transference mechanism is configured to move the ultrasound transducer assembly along the one or more predetermined paths within the container. The one or more predetermined paths are one or more programmatically defined paths. The transference mechanism is configured to move the ultrasound transducer assembly along a spiral path about the longitudinal axis of the bony anatomy. The transference mechanism is configured to generate position data indicative of a position of the ultrasound transducer assembly. To determine a position corresponding to each of the data sets, the instructions, when executed by the one or more processors, cause the one or more processors to access information about the one or more predetermined paths and information about a speed of the ultrasound transducer assembly along the one or more predetermined paths and determine the position corresponding to each of the data sets based on the speed of the ultrasound transducer assembly and the information about the one or more predetermined paths. The transference mechanism is configured to maintain a substantially constant distance between the ultrasound transducer assembly and the longitudinal axis along the one or more predetermined paths.

In another general aspect, a system for ultrasound scanning includes an ultrasound transducer assembly configured to generate ultrasound scan data indicative of surface features of bony anatomy. The system includes a first reference attachable to the ultrasound transducer assembly and configured to move with the ultrasound transducer assembly when attached to the ultrasound transducer assembly. The system includes a second reference configured to be removably and extracutaneously secured to a patient. The spatial tracking device configured to communicate with the first reference and the second reference. The system includes one or more processors and one or more data storage devices storing instructions that, when executed by the one or more processors, cause the one or more processors to perform operations. The operations include receiving the ultrasound scan data, the ultrasound scan data comprising data sets acquired at different positions of the ultrasound transducer assembly relative to the bony anatomy from a direction substantially orthogonal to the bony anatomy. The operations include receiving first position data from the first reference or the spatial tracking device, the first position data indicating different positions of the first reference during acquisition of the ultrasound scan data while the first reference is attached to the ultrasound transducer assembly. The operations include receiving second position data from the second reference or the spatial tracking device, the second position data indicating different positions of the second reference during acquisition of the ultrasound scan data while the second reference is extracutaneously secured to a patient. The operations include associating each data set of the ultrasound scan data with the position of the first reference and the position of the second reference when the data set was acquired. The operations include generating a three-dimensional representation of the bony anatomy based on the ultrasound scan data using the positions of the first reference and the second reference that are associated with the data sets to compensate for movement of the patient during acquisition of the ultrasound scan data.

Implementations may include one or more of the following features. For example, the system includes a third reference configured to be removably and extracutaneously secured to a patient. The operations further comprise: receiving third position data from the third reference or the spatial tracking device, the third position data indicating different positions of the third reference during acquisition of the ultrasound scan data while the third reference is extracutaneously secured to the patient; and associating each data set of the ultrasound scan data with the position of the third reference when the data set was acquired. Generating the three-dimensional representation of the bony anatomy based on the ultrasound scan data includes generating the three-dimensional representation of the bony anatomy based on the ultrasound scan data using the positions of the first reference, the second reference, and the third reference that are associated with the data sets to compensate for movement of the patient during acquisition of the ultrasound scan data.

Implementations may include one or more of the following features. For example, receiving the second position data includes receiving second position data indicating different positions of the second reference while the second reference moves with a tibia of the patient. Receiving the third position data includes receiving third position data indicating different positions of the third reference while the third reference moves with a femur of the patient. Generating the three-dimensional representation of the bony anatomy includes generating a three-dimensional representation of a knee joint comprising portions of the tibia and the femur. The scan data includes data sets acquired from each of a medial side, a lateral side, a posterior side, and an anterior side of the knee. The scan data includes data sets acquired while the knee is in multiple different angles of flexion. The ultrasound transducer assembly is configured to produce ultrasound at a frequency of between of 5 MHz to 10 MHz. Each of the data sets of the scan data represents a transverse image of a portion of the bony anatomy. Generating the representation of the bony anatomy includes generating a three-dimensional surface using on at least one of a level set algorithm, a radial basis function, a marching cubes algorithm, or interpolation using a voxel array. Generating the representation of the bony anatomy includes registering at least a plurality of the data sets relative to each other based on anatomical features indicated in the data sets. Registering at least a plurality of the data sets relative to each other based on anatomical features indicated in the data sets includes registering the plurality of data sets relative to each other based on portions of the plurality of data sets corresponding to one or more of a bone edge, a condyle, an epicondyle, and a tibial tuberosity. The first reference is a fiducial, and the spatial tracking device includes an optical detector. The first reference is an electromagnetic field sensor, and the spatial tracking device includes an electromagnetic field generator. The first reference is a radiofrequency sensor, and the spatial tracking device includes a radiofrequency transmitter. The spatial tracking device is a mechanical linkage configured to measure movement, and the first reference is an attachment element attached to the mechanical linkage. The system includes one or more of a brace, a sleeve, a band, a strap, a belt, a clip, an adhesive, or an elastic configured to extracutaneously attach the second reference to the anatomy of the patient. Generating the three-dimensional representation of the bony anatomy includes: identifying points that correspond to surfaces of the bony anatomy based on the scan data; registering the identified points in a coordinate reference system to form a three-dimensional point cloud; and determining a three-dimensional surface corresponding to the bony anatomy based on the point cloud.

The details of one or more implementations are set forth in the accompanying drawings and the description below. Other features, objects, and advantages of the disclosure will be apparent from the description and drawings, and from the claims.

### BRIEF DESCRIPTION OF THE DRAWINGS

FIG. 1A is a diagram illustrating a system for ultrasound scanning.

FIG. 1B is a cross-sectional view of a limb during an ultrasound scan.

FIG. 1C is a diagram of an ultrasound transducer assembly and a representation of scan data.

FIG. 1D is a diagram illustrating a path of the ultrasound transducer assembly relative to a limb.

FIG. 2A is a diagram of another system for ultrasound scanning.

FIGS. 2B-2E are diagrams of the system of FIG. 2A with various spatial tracking components.

FIG. 2F is a diagram of a process for providing feedback to an operator of the system of FIG. 2A.

FIG. 3 is a diagram of another system for ultrasound scanning.

FIGS. 4A and 4B are diagrams of another system for ultrasound scanning.

FIGS. 5A to 5D are diagrams of alternative paths that an ultrasound transducer assembly can take during ultrasound scanning.

FIG. 6 is a diagram of another system for ultrasound scanning.

FIG. 7A is a diagram of another system for ultrasound scanning.

FIG. 7B is a cross-sectional view of a cuff of the system of FIG. 7A.

FIG. 7C is a cross-sectional view and schematic view of the system of FIG. 7A.

FIG. 7D is a cross-sectional view of the system of FIG. 7A.

FIG. 7E is a cross-sectional view and schematic view illustrating a transference mechanism.

FIG. 8 is a flow diagram of a process for creating a surgical guide using ultrasound imaging data.

### DETAILED DESCRIPTION

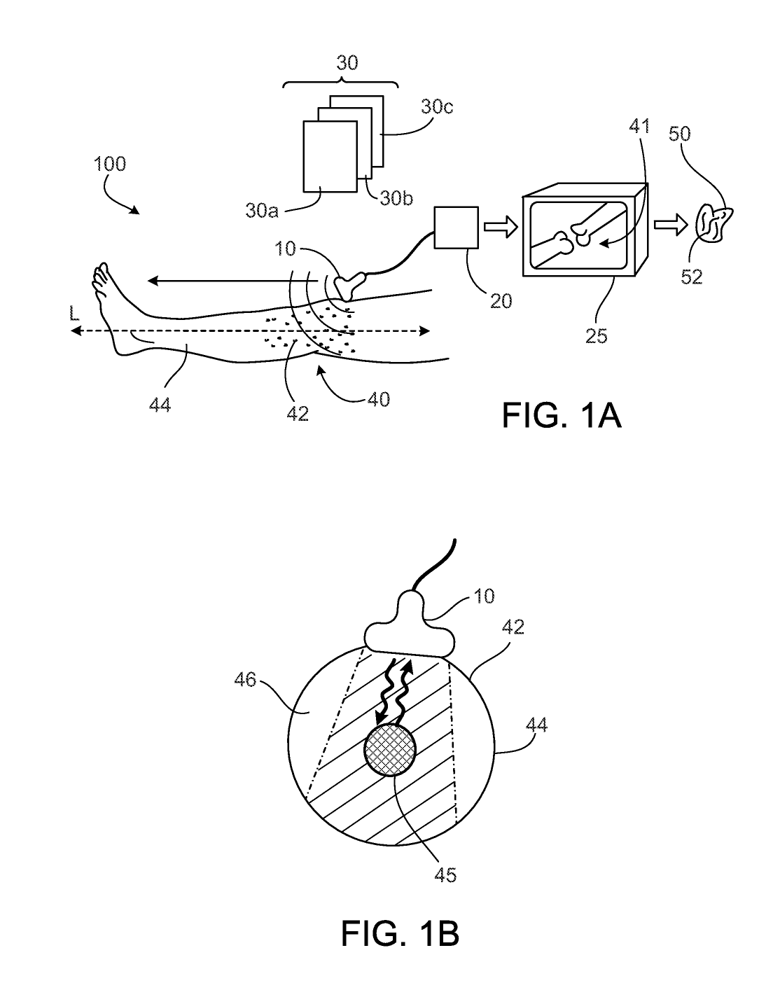

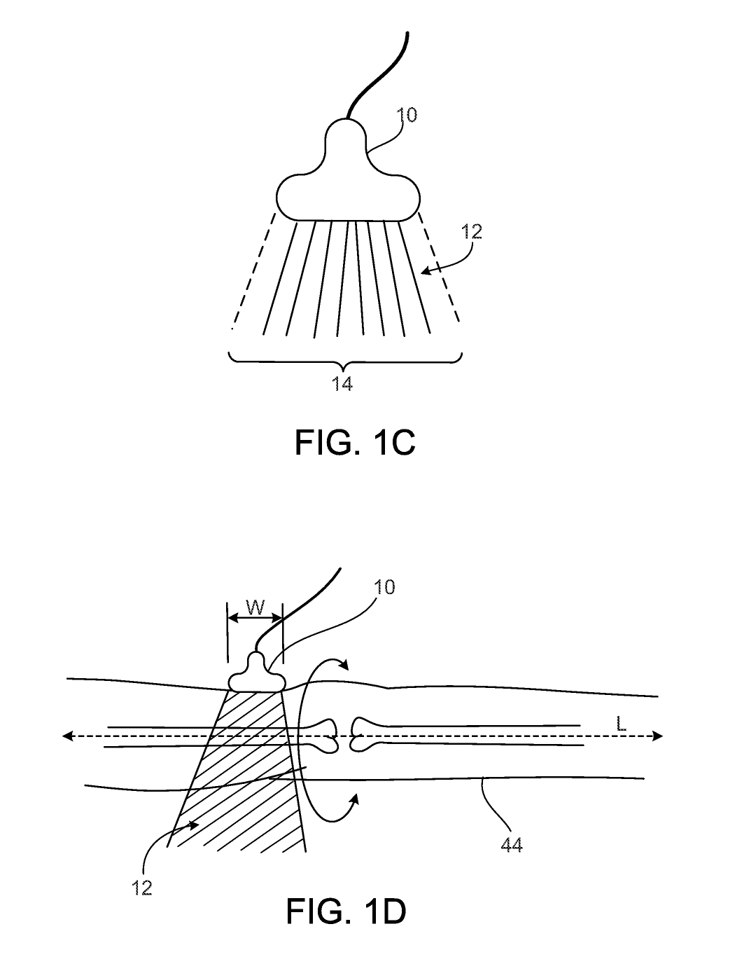

Referring to FIG. 1A, a system 100 for ultrasound scanning can be used to generate images of bony tissue. The system 100 includes an ultrasound transducer assembly 10 that provides ultrasound scan data that is received by a control unit 20 and/or another processing system 25. Using the system 100, an operator can acquire ultrasound imaging scan data 30 for an anatomical region of interest, such as a patient's joint. The joint can be, for example, a knee 40 or any other joint. The ultrasound scan data 30 is then processed and used to generate a digital model 41 or other three-dimensional (3D) representation of the knee 40. The digital model 41 can be used for medical diagnosis and pre-operative planning. For example, the digital model 41 can be used to create a surgical guide 50 with patient-specific contours 52 for use in arthroplasty or other surgical procedures. As another example, the digital model 41 can be used to shape or assemble an implant with patient-specific characteristics.

Ultrasound imaging data can be used instead of CT and MRI scans to create a 3D representation of a bone or joint. Ultrasound imaging is safe, non-invasive, and does not expose patients to ionizing radiation. Ultrasound imaging can be performed with portable, low-cost equipment as a practical and valuable alternative to other medical imaging methods, to image both soft and hard tissues. The small size and relatively low cost of ultrasound equipment compared to CT and MRI equipment permits expanded access to imaging technology and permits imaging in areas where more costly scanning options are not available. Also, unlike CT scans, ultrasound scans do not expose patients to ionizing radiation.

The ultrasound transducer assembly 10 includes one or more ultrasound transducers, for example, a transducer array. The frequency of ultrasound emitted and detected by the ultrasound transducer assembly 10 can be selected such that the ultrasound propagates through soft tissue and reflects from the surface of bone. For example, ultrasound with a frequency between 5 MHz and 10 MHz may be used. In some implementations, a lower frequency of ultrasound can be used to penetrate through bone to a desired depth, for example, to permit imaging through the patella.

To acquire the ultrasound scan data 30 using the system 100, a human operator first applies an ultrasound-conductive medium, such as a gel 42, to the knee 40. The operator then contacts the ultrasound transducer assembly 10 to the gel 42 on the knee 40. The operator moves the ultrasound transducer assembly 10 relative to the knee 40 while maintaining contact between the ultrasound transducer assembly 10 and the gel 42.

Referring to FIG. 1B, a cross-sectional view of the leg 44 illustrates the interaction of the ultrasound transducer assembly 10 with the leg 44. To perform a scan, the ultrasound transducer assembly 10 emits ultrasound waves, which are transmitted through the gel 42 and the soft tissue 46 of the leg 44. The ultrasound transducer assembly 10 detects ultrasound waves that reflect from the bone 45 of the leg 44, for example, from the surface of the bone 45. Because the propagation speed of the ultrasound waves is known, the delay between transmission of ultrasound and detection of the reflection is indicative of the distance between the ultrasound transducer assembly 10 and the bone.

Referring to FIG. 1C, the ultrasound transducer assembly 10 can direct ultrasound along different axes of a planar scan area 12. Ultrasound can be emitted along each of the different axes 14 in a sequence, for example, sweeping from one side to another. After transmission along a particular axis 14, the ultrasound reflections that are detected in response represents a collection of pixels or a single measurement. Each data set 30a-30c can include pixels or measurements along each of the axes 14 in the scan area 12. As a result, each data set 30a-30c can include data for a transverse image, or “slice” of a bone, where the scan area 12 intersects the bone. Information about multiple depths may be acquired along each axis, providing a two-dimensional view through some tissues. For example, the timing of different echoes in response to transmission along an axis can provide information about different tissue interfaces and different tissue types.

The data sets 30a-30c, or image slices, or can be acquired during movement of the ultrasound transducer assembly 10 relative to a joint or bone. The propagation speed of the ultrasound is much faster than the speed at which an operator moves the ultrasound transducer assembly 10 relative to a joint, and thus the time required to acquire a data set 30a-30c is relatively short. As a result, all of the data for a given data set 30a-30c can be acquired while the ultrasound transducer assembly 10 is in a substantially consistent position relative to the joint, even though the ultrasound transducer assembly 10 may be in motion relative to the joint.

Referring again to FIG. 1A, as the operator moves the ultrasound transducer assembly 10, the control unit 20 controls the ultrasound transducer assembly 10 to perform multiple scans, for example, to perform scans at fixed time intervals. The control unit 20 receives and stores the ultrasound scan data 30, and records the sequence in which each data set 30a-30c is generated.

Scans are also made along multiple trajectories to obtain a complete view around the knee 40. For example, with the patient's leg 44 in extension, the operator can move the ultrasound transducer assembly 10 in a direction substantially parallel to a longitudinal axis, L, of the leg 44, with the plane of the scan area 12 oriented transverse to the longitudinal axis, L. To acquire data about multiple surfaces of the knee 40, scan data 30 can be acquired while moving the ultrasound transducer assembly 10 along multiple substantially linear paths, for example, along a medial side, a lateral side, a posterior side, and an anterior side of the knee 40.

Referring to FIG. 1D, other orientations of the ultrasound transducer assembly 10 and other scanning trajectories can be used. For example, an operator can orient the ultrasound transducer assembly 10 such that the majority of, or all of, the scan area 12 intersects bone. For example, a width, W, of the scan area 12 can be oriented substantially parallel to the longitudinal axis, L, of the leg 44 and the scan area 12 intersects the bone. The operator can then move the ultrasound transducer assembly 10 about the circumference of the leg 44 in a circular or a spiral path.

The knee 40 can be scanned with the leg 44 in extension or at a desired angle of flexion, for example, at 90 degrees flexion. The leg 44 can be substantially immobilized during multiple passes over the knee 40 to facilitate correlation of the data from different scan paths.

In some implementations, multiple sets of scan data are acquired at different angles of flexion of the knee 40 to expose different articular surfaces of the knee 40. For example, with the knee 40 in extension, scan data 30 can be acquired during movement of the ultrasound transducer assembly 10 along each of the medial side, lateral side, posterior side, and anterior side of the knee 40. The knee can then be placed in 90 degrees flexion, and additional scan data 30 can be acquired while the ultrasound transducer assembly 10 is moved along the medial side, lateral side, posterior side, and anterior side of the knee 40.

Using the ultrasound scan data 30, a processing system 25 (which in some implementations includes the control unit 20) determines contours of a 3D surface that represents the outer surface of the knee 40. In some implementations, as shown in the system 100, the position of the ultrasound transducer assembly 10 is not tracked during scanning. The processing system 25 applies image registration algorithms to the scan data 30 to “stitch” together the images into a single surface. As an example, surface-fitting techniques can be used to generate surface that span regions described by multiple data sets 30a-30c. Points that correspond to bone surfaces can be identified in each data set 30a-30c. Collectively, the various points can form a “point cloud” in a 3D reference frame. The surface of the bone can be modeled from the point cloud using parameterized surface fitting techniques to extract shape and surface-texture information. For example, the Paraform software package, provided by Paraform, Inc. of 3052 Bunker Hill, Santa Clara, Calif., can be used. Techniques described in U.S. Pat. Nos. 6,271,856, 6,256,038, and 6,256,039, all of which are incorporated herein by reference in their entirety, can be used.

When scan data 30 is acquired with unconstrained or “freehand” scanning, the speed of movement of the ultrasound transducer assembly 10, the direction of movement of the ultrasound transducer assembly 10, the angle of the ultrasound transducer assembly 10 relative to the leg 44, and other scan characteristics are not consistent. To compensate for variations or inconsistencies during scanning, the processing system identifies commonalities among the data in different data sets 30a-30c. Different data sets 30a-30c are registered relative to each other based on similarities in the surface features detected. Groups of data sets 30a-30c can be used to calculate bone surfaces, and different surfaces can be registered based on shared overlapping regions or features. In some implementations, particular anatomical features that are discernable from the data sets 30a-30c are used to align surfaces relative to each other. For imaging of the knee 40, for example, bone edges, condyles, epicondyles, a tibial tuberosity, and other features may be used to register one data set 30a-30c or surface relative to another data set 30a-30c or surface.

Other techniques can be used to generate a 3D surface from data sets 30a-30c that represent different image slices of the knee 40. For example, a level set algorithm, a radial basis function, a marching cubes algorithm, and interpolation using a voxel array can be used to generate a 3D surface. Noise reduction and other image processing algorithms can also be applied.

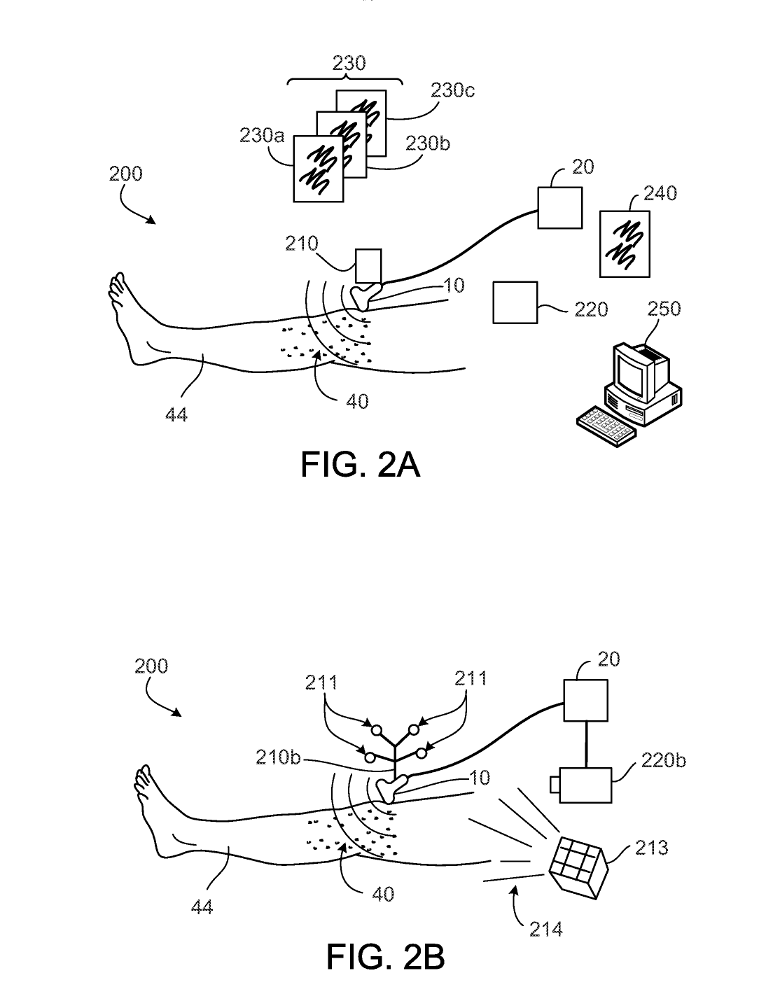

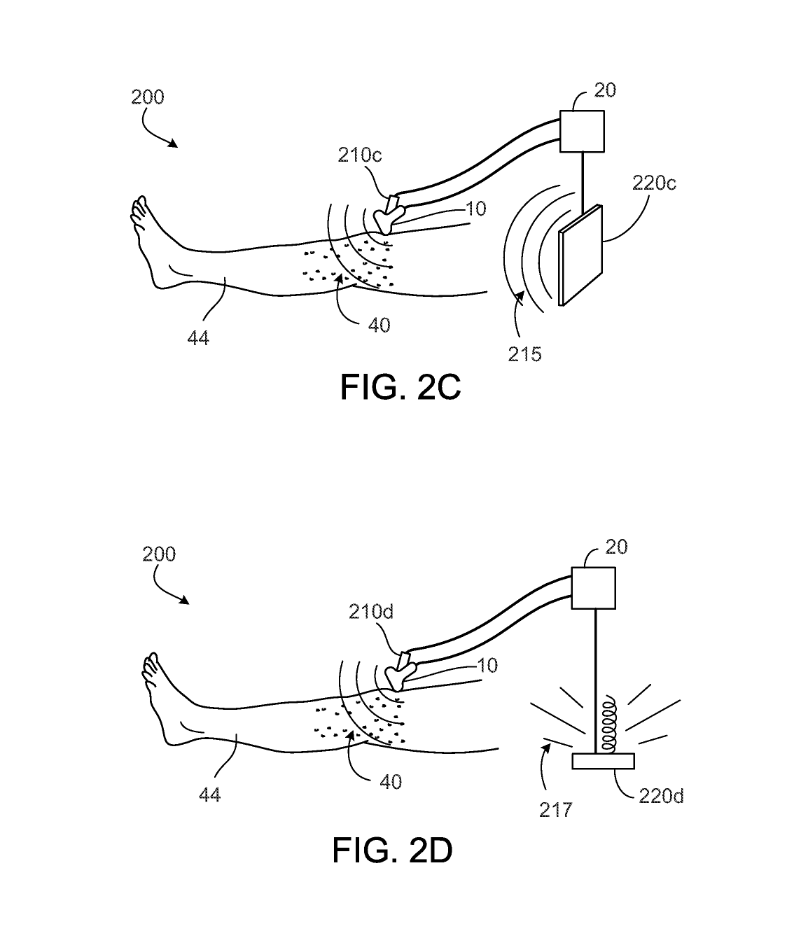

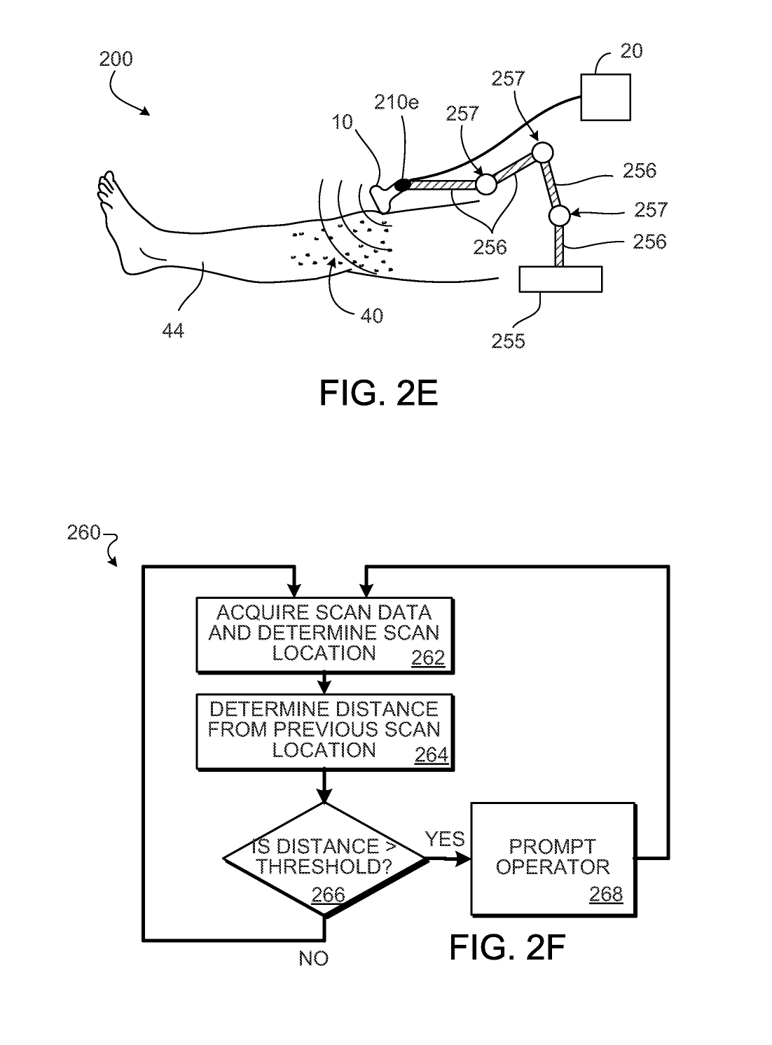

Referring to FIG. 2A, an ultrasound scanning system 200 includes the ultrasound transducer assembly 10, the control unit 20, a reference 210, and a spatial tracking device 220. The system 200 tracks the position of the ultrasound transducer assembly 10 during scanning to facilitate construction of a 3D representation using ultrasound scan data produced by the ultrasound transducer assembly 10. When ultrasound scan data is acquired, the control unit 20 also receives information about the position of the ultrasound transducer assembly 10. For example, the control unit 20 can receive information about both a location and an orientation of the ultrasound transducer assembly 10 relative to a reference device, or relative to a coordinate reference frame.

In the manner described above, an operator moves the ultrasound transducer assembly 10 relative to an anatomical region of interest, such as the knee 40, to perform a scan. The knee 40 is immobilized during scanning. As scanning proceeds, the ultrasound transducer assembly 10 generates ultrasound scan data 230 that is indicative of surface features of bony anatomy of the knee 40. The ultrasound scan data 230 includes data sets 230a-230c acquired at different positions of the ultrasound transducer assembly 10 relative to the knee 40.

The reference 210 is attachable to the ultrasound transducer assembly 10. When attached to the ultrasound transducer assembly 10, the reference 210 moves with the ultrasound transducer assembly 10. The reference 210 can be attached to the ultrasound transducer assembly 10 with a band, strap, bolt, screw, adhesive, clamp, or other fastener. The reference 210 can be attached at a known position and orientation relative to the ultrasound transducer assembly 10, for example, at a known positional offset from a transducer surface of other landmark. In some implementations, rather than attach the reference at a known position, a registration process can be used to determine a positional offset between the position of the reference 210 and a transducer surface or other landmark of the ultrasound transducer assembly 10. The reference may be calibrated or registered in a coordinate reference frame before ultrasound scanning begins.

The spatial tracking device 220 communicates with the reference 210, such that the reference 210, the spatial tracking device 220, or both, generate data that indicates the position of the reference 210 in a coordinate reference system. The reference 210 and the spatial tracking device 220 can communicate through any of several types of operative connections, described further below, including, mechanical, electromagnetic, and optical communication links.

The control unit 20 performs a number of operations. The control unit 20 includes one or more processors and one or more data storage devices storing instructions that, when executed by the one or more processors, cause the one or more processors to perform the various operations. For example, the control unit 20 sends signals that cause the ultrasound transducer assembly 10 to initiate ultrasound scans at defined intervals.

The control unit 20 also receives data and processes the received data. The control unit 20 receives the ultrasound scan data 230, which includes the data sets 230a-230c acquired at different positions of the ultrasound transducer assembly relative to the bony anatomy. Each of the data sets 230a-230c can be acquired with the ultrasound transducer assembly oriented substantially orthogonal to the bony anatomy. For example, during scanning, the operator can orient the ultrasound transducer assembly 10 such that the scan area 12 is substantially transverse to the longitudinal axis, L, of the leg 44. Alternatively, the operator can orient the ultrasound transducer assembly 10 such that the scan area 12 is parallel to or includes the longitudinal axis, L.

The control unit 20 receives position data 240 from the reference 210 or the spatial tracking device 220. The position data 240 indicates different positions of the reference 210 during acquisition of the ultrasound scan data 230 while the reference is attached to the ultrasound transducer assembly 10. The control unit 20 associates each data set 230a-230c of the ultrasound scan data 230 with the position of the reference 210 when the ultrasound transducer assembly 10 detected the data in the data set 230a-230c. The control unit 20 can form the association by, for example, storing data that indicates the positions corresponding to each data set 230a-230c. The control unit 20 can communicate with the reference 210 and/or the spatial tracking device 220 over a wired or a wireless communication link.

With the position data 240 associated with each data set 230a-230c, the control unit 20 or another processing system 250 can register the data sets 230a-230c relative to each other in a coordinate reference plane. As a result, the processing system 250 can generate a 3D surface that represents the bony anatomy of the knee 40, taking into account variation in the positions of ultrasound transducer assembly 10. Because the position of the ultrasound transducer assembly 10 is tracked, the operator is not limited to a substantially linear scan path. Multiple scans, for example, scans from different perspectives or orientations relative to the knee 40, can be registered to each other to form a complete, 360-degree view of the knee 40.

The control unit 20 can synchronize the acquisition of the position data 240 and the scan data 230. For example, the control unit 20 can send signals that initiate each scan performed by the ultrasound transducer assembly 10 and send signals to request a current position of the reference 210. Where position data 240 is provided continuously, the control unit 20 can sample the position data 240 to acquire data that corresponds to the time and position of each generated data set 230a-230c.

Referring to FIG. 2B, the system 200 is illustrated in a configuration for optical tracking. A fiducial 210b is used as the reference 210, and an optical detector 220b, such as a camera, is used as the spatial tracking device 220. The fiducial 210b includes reflective elements 211, such as metallic surfaces or spheres that reflect infrared light. The reflective elements 211 can be arranged to define a plane. A light source 213 directs infrared light 214 to the fiducial 210b, and the optical detector 220b detects reflections from the fiducial 210b. The optical detector 220b transmits information about the reflected light to the control unit 20, which uses triangulation or other techniques to determine the position of the fiducial 210a relative to the optical detector 220b, which can remain in a fixed position relative to the knee 40 during ultrasound scanning.

Referring to FIG. 2C, the system 200 is illustrated in a configuration for spatial tracking using electromagnetic fields. A magnetic sensor 210c, for example, an electromagnetic field sensor including one or more inductive coils, is used as the reference 210, and an electromagnetic field generator 220c is used as the spatial tracking device 220. The magnetic sensor 210c can include one or more of, for example, a Hall effect sensor, a fluxgate magnetic field sensor, and a magneto-resistive sensor, in addition to or instead of an inductive coil.

The electromagnetic field generator 220c generates electromagnetic field patterns 215 that are detected by the magnetic sensor 210c. When the magnetic sensor 210c detects sufficient electromagnetic field energy, the magnetic sensor 210c produces signals indicating the position of the magnetic sensor 210c relative to the electromagnetic field generator 220c, which can remain in a fixed position relative to the knee 40 during ultrasound scanning. The control unit 20 receives the signals from the magnetic sensor 210c and information about the electromagnetic fields produced by the electromagnetic field generator 220c, and uses the information to detect changes in position of the magnetic sensor 210c.

The magnetic sensor 210c, the electromagnetic field generator 220c, and the control unit 20 can include features as described in WIPO International Publication Nos. WO2008/106593 and WO2009/108214, each of which is incorporated herein by reference in its entirety, and as described in U.S. patent application Ser. Nos. 12/758,747 and 12/768,689, each of which is incorporated herein by reference in its entirety.

Referring to FIG. 2D, the system 200 is illustrated in a configuration for spatial tracking using radiofrequency transmissions. A radiofrequency sensor 210d, for example, a sensor that includes one or more radiofrequency antennas, is used as the reference 210, and a radiofrequency transmitter 220d is used as the spatial tracking device 220. The radiofrequency transmitter 220d transmits patterns of radiofrequency transmissions 217 that are detected by the radiofrequency sensor 210d. When the radiofrequency sensor 210d detects sufficient radiofrequency energy, the radiofrequency sensor 210d produces signals indicating the position of the radiofrequency sensor 210d relative to the radiofrequency transmitter 220d, which can remain in a fixed position relative to the knee 40 during ultrasound scanning. The control unit 20 receives the signals from the radiofrequency sensor 210d and information about the transmissions of the radiofrequency transmitter 220d, and uses the information to detect changes in position of the radiofrequency sensor 210d.

Referring to FIG. 2E, the system 200 is illustrated in a configuration for spatial tracking using mechanical communication. A mechanical linkage 220e, such as an articulating arm; is used as the spatial tracking device 220. The mechanical linkage 220e is anchored to a base 255 that can remain in a fixed position relative to the knee 40 during ultrasound scanning. The mechanical linkage 220e can include multiple segments 256 connected by moveable joints 257. Each joint can permit movement in one or more degrees of freedom. The joints can include, for each degree of freedom permitted, an encoder or other position-tracking mechanism. Mechanical encoders, optical encoders, and other position detection mechanisms can be used. Data indicating the positions of the various joints is transmitted to the control unit 20.

An attachment element 210e, for example, a fastener located at an end of the mechanical linkage 220e, is used as the reference 210. The attachment element 210e attaches to the ultrasound transducer assembly 10, such that the ultrasound transducer assembly 10 is mechanically connected to the base through the mechanical linkage 220e. During ultrasound scanning, the joints of the mechanical linkage 220e move, permitting the operator to move the ultrasound transducer assembly 10 while it is attached to the mechanical linkage 220e. The control unit 20 uses the data that indicates the positions of the joints to determine changes in the position of the ultrasound transducer assembly 10 from one scan location to the next scan location.

Referring to FIG. 2F, the control unit 20 can provide feedback to an operator while acquiring ultrasound scan data according to a process 260. The feedback can indicate to an operator, for example, that the change in distance of the ultrasound transducer assembly 10 between acquired data sets is excessive, and thus, that an operator should move the ultrasound transducer assembly more slowly.

The control unit 20 acquires scan data and determines a scan location where the scan data was produced (262). For example, the location can be a location of a reference 210 that moves with the ultrasound transducer assembly 10 at the time the scan data was generated by the ultrasound transducer assembly 10.

The control unit 20 determines a distance between the determined scan location and a previous scan location (264). For example, the control unit 20 can calculate a distance between the scan location for the most recent data set and a scan location for the data set received immediately prior.

The control unit 20 determines whether the distance is greater than a threshold (266). The threshold can be a predetermined value that indicates a maximum desired change in distance between acquisition positions. In some implementations, the threshold may represent a change in position along a single axis or along multiple axes. If the distance is greater than the threshold, the control unit prompts the operator, for example, providing a message or alarm indicating that the scan locations are too far apart to achieve a desired level of resolution. Scanning can then resume, for example, automatically, after a predetermined delay period, or in response to interaction of the operator with the control unit 20.

In some implementations, the control unit 20 determines changes in the orientation of the ultrasound transducer assembly 10 are also determined and compared to threshold values. If the orientation is determined to change beyond a predetermined level, for example, a threshold change in angle, an alarm or other message can be provided to the operator.

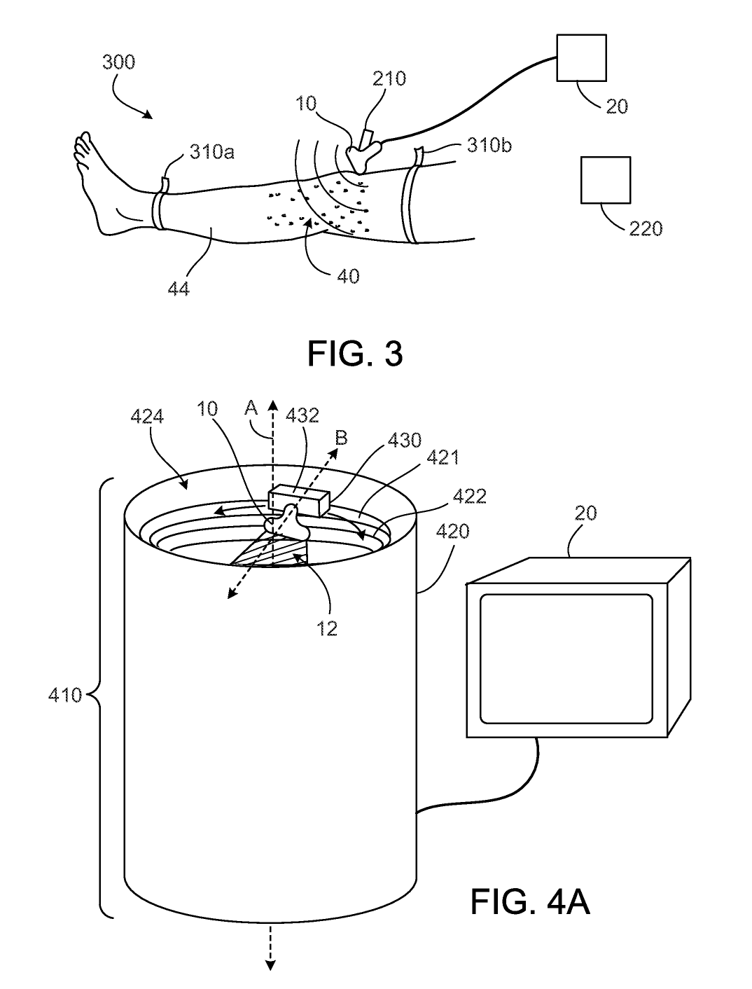

Referring to FIG. 3, a system 300 for ultrasound scanning includes the ultrasound transducer assembly 10, the control unit 20, the reference 210, the spatial tracking device 220, and one or more additional references 310a, 310b. The additional references 310a, 310b are attachable to a patient and can be used to detect movement of the patient's leg 44 during scanning. Data about the positions of the references 310a, 310b during scanning can be used to compensate for inadvertent or undesired movement of the leg 44. The data can also facilitate generation of a 3D representation of the knee 40 using scans of different positions of the knee 40, for example, to correlate data generated while the knee 40 is in different angles of flexion. Although, two additional references 310a, 310b are illustrated in FIG. 3, the system 300 can include only one reference for tracking the position of the patient, or may include three or more references to track the position of the patient.

The references 310a, 310b can attach to the patient in a non-invasive manner, for example, with an extracutaneous fastener such as a brace, a sleeve, a band, a strap, a belt, a clip, an adhesive, or an elastic. The references 310a, 310b can be skin-mounted sensors or markers that attach directly to the skin. In some implementations, anatomical features or other landmarks can be used as references 310a, 310b. The references 310a, 310b can each be attached securely such that sliding and tilting motions relative to the leg 44 are impeded. The references 310a, 310b can be spaced apart on opposite sides of the knee 40. One reference 310a can be attached to the lower portion of the leg 44, for example, about the shin or slightly above the ankle, such that the reference 310a moves with a tibia of the leg 44. The other reference 310b can be fastened at the thigh such that the reference 310b moves with a femur of the leg 44. The references 310a, 310b are also spaced apart from the knee 40 to avoid interference with the ultrasound transducer assembly 10 during scanning.

The position of the references 310a, 310b can be determined in the same manner that the position of the reference 210 is determined. Accordingly, the references 310a, 310b may each include a fiducial, an electromagnetic field sensor, a radiofrequency sensor, or an attachment element of a mechanical linkage. A registration process can be used to establish the positions of the references 210, 310a, 310b in a common reference frame. As ultrasound scanning proceeds, the control unit 20 receives data about the positions (e.g., location and orientation) of the references. The control unit 20 associates ultrasound scan data generated by the ultrasound transducer assembly 10 with the corresponding positions of the references 310a, 310b when the scan data was generated. The position data can then be used to calculate the position of the ultrasound transducer assembly 10 relative to the position of the knee 40 for each data set.

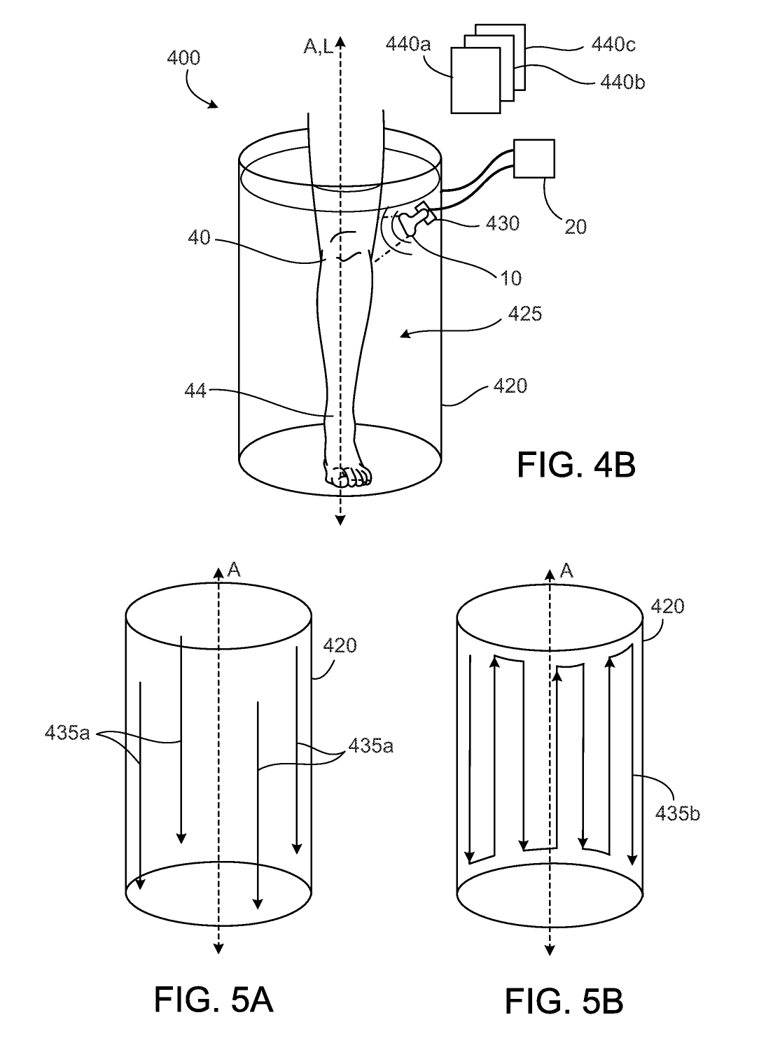

Referring to FIG. 4A, an ultrasound scanning system 400 includes the ultrasound transducer assembly 10, the control unit 20, and a scanning assembly 410 that performs automated scanning. The scanning assembly 410 includes a container 420 that can hold an ultrasound-conductive medium, and a transference mechanism 430 for the ultrasound transducer assembly 10. The transference mechanism 430 moves the ultrasound transducer assembly 10 within the container 420 along a controlled path. The ultrasound transducer assembly 10 generates ultrasound scan data at different positions along the path, and transmits the generated scan data to the control unit 20. The transference mechanism 430 can maintain a desired orientation of the ultrasound transducer assembly 10 along the path, for example, maintaining a beam direction that is substantially orthogonal to an axis.

The transference mechanism 430 provides automated movement of the ultrasound transducer assembly 10. The transference mechanism 430 can include a moveable element 432 that attaches to and moves with the ultrasound transducer assembly 10. The moveable element 432 can move along a track 422 defined on an inner surface 421 of the container 420. The track 422 can define one or more predetermined paths that extend in different directions, as described further below. The track 422 can be defined by the container 420 at an inner surface 421 of the container 420. The track 422 can be defined by rails, grooves, threads, or other features. In addition to, or as alternative to a mechanical track, the transference mechanism 430 can include a support frame (not shown) and motorized elements that move the moveable element 432 to desired positions or in desired directions within the container 420. The control unit 20, or a control system of the transference mechanism 430, can be programmed to move the moveable element 432 along a variety of operator-defined paths.

The transference mechanism 430 can communicate with the control unit 20, for example, to receive signals that control movement of the ultrasound transducer assembly 10 and/or to provide data indicative of the position of the ultrasound transducer assembly 10. Movement of the ultrasound transducer assembly 10, for example, the speed and direction along the path, can be controlled by the control unit 20 or a control system embedded in the transference mechanism 430. The position of the ultrasound transducer assembly 10 can be determined by the control unit 20, for example, based on a known speed of the ultrasound transducer assembly 10 and known characteristics of the path. In addition, or as an alternative, the transference mechanism 430 can include encoders or other mechanisms to measure a position of the ultrasound transducer assembly 10.

The container 420 defines an opening 424 that admits a subject to be scanned, such as the knee 40. The container 420 defines an axis, A, through the opening 424. In some implementations, the transference mechanism 430 orients the ultrasound transducer assembly 10 such that a central axis, B, through the scan area 12 intersects the axis, A, or extends within a predetermined distance from the axis, A. The transference mechanism 430 can maintain a predetermined relationship between the axes, A and B, along a majority of, or along the entirety of, a path while ultrasound scan data is acquired. As a result, the transference mechanism 430 can control the beam direction of the ultrasound transducer assembly 10 to achieve a desired level of resolution.

The transference mechanism 430 can maintain a desired beam direction relative to the axis, A, at multiple positions about the axis, and while moving the ultrasound transducer assembly 10 circumferentially about the axis, A, in a direction parallel to the axis, A, or in other directions. While maintaining the desired relationship between the axes, A and B, the transference mechanism 430 can also maintain other aspects of the orientation of the ultrasound transducer assembly 10, for example, maintaining the scan area 12 transverse to the axis, A, parallel to the axis, A, or at another angle relative to the axis, A. The transference mechanism 430 can move the ultrasound transducer assembly 10 with a constant speed or other controlled speed, and can maintain a constant distance between the ultrasound transducer assembly 10 and the axis, A. In some implementations, the container 420 has a substantially cylindrical shape, which can facilitate a constant distance between the ultrasound transducer assembly 10 and the axis, A.

Referring to FIG. 4B, to use the ultrasound scanner, the knee 40 or other region of interest is positioned in the container 420 and submerged in an ultrasound conductive medium 425. For example, to acquire an image of a patient's left knee 40, the patient steps into the container 420 with her left leg 44 so that the patient is standing with the left leg 44 in the container 420. Before or after the patient steps into the container 420, the container 420 is filled with the ultrasound-conductive medium 425 to a sufficient level that the knee 40 is immersed. The ultrasound-conductive medium 425 can be, for example, water or hydrogel. The patient may be supported, or the patient's leg 44 substantially immobilized, such that a substantially consistent position of the leg 44 is maintained during scanning.

With the knee 40 immersed, the operator initiates ultrasound scanning. The transference mechanism 430 moves the ultrasound transducer assembly 10 along a path within the container 420. For example, the transference mechanism 430 can move the ultrasound transducer assembly 10 in a direction parallel to the axis, A, or in a spiral direction about the axis, A. Other paths can also be used. The path can include segments that position the ultrasound transducer assembly 10 to acquire data at different perspectives or views of the knee 40, for example, to acquire image data while facing different sides of the knee 40.

The movement of the transference mechanism 430 can be controlled by signals from the control unit 20 or an embedded control system of the transference mechanism 430. The ultrasound transducer assembly 10 and the knee 40 are both immersed in the ultrasound conductive medium 425, and therefore the ultrasound transducer assembly 10 is not required to contact the knee 40. At various positions relative to the knee 40, for example, at different points in time while the ultrasound transducer assembly 10 moves along the path, the ultrasound transducer assembly 10 acquires a data set 440a-440c that includes data for an image slice of the knee 40.

The control unit 20 receives the data sets 440a-440c and calculates the corresponding position of the ultrasound transducer assembly 10. The different data sets 440a-440c can be acquired at a defined spacing relative to each other. For example, the ultrasound transducer assembly 10 can move along segments of the path at a constant speed, and data sets 440a-440c can be acquired at a consistent time interval, resulting in a consistent distance between scan locations. A regular spacing of images can facilitate generation of a 3D surface based on the images. The control unit 20 can access information about the shape and size of the path to register the data sets 440a-440c relative to each other. In some implementations, the ultrasound transducer assembly 10 records data at 0.6 second intervals, resulting in data for approximately 200 image slices over about two minutes.

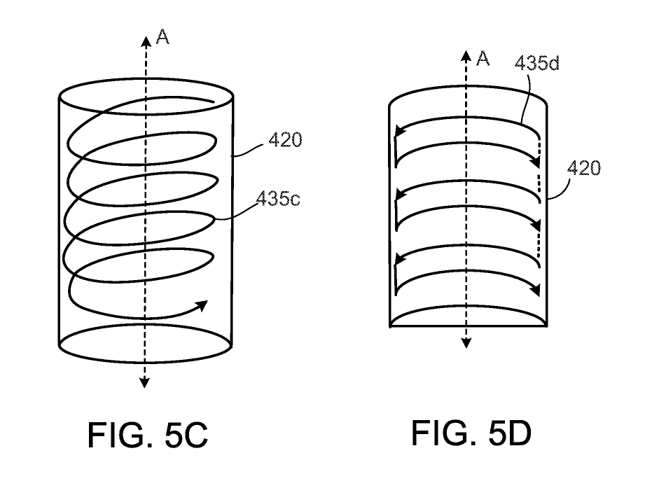

Referring to FIGS. 5A to 5D, the transference mechanism 430 can move the ultrasound transducer assembly 10 along one or more of various paths 435a-435d, respectively, within the container 420. Each of the paths 435a-435d can maintain an equal distance between the ultrasound transducer assembly 10 and the axis, A, along positions where ultrasound scan data is acquired. Each of the paths 435a-435d positions the ultrasound transducer assembly 10 at different perspectives about the axis, A, which permits ultrasound scanning

The paths 435a-435d can be mechanically defined by features of the container 420 and the transference mechanism 430. Alternatively, the paths 435a-435d can be programmatically defined by the control unit 20 or other control systems. Ultrasound scanning occurs at different positions along the paths 435a-435d, at particular portions of the paths or continuously along the paths. Movement along the paths 435a-435d can occur in the directions illustrated, or in the opposite directions. The paths 435a-435d may extend along the length of the container 420 or over a portion of the container 420 that is of interest. Accordingly, the paths 435a-435d can be compressed or expanded in the direction of the axis, A, to provide scanning over a particular region of interest.

Referring to FIG. 5A, the path 435a includes motion substantially parallel to the axis, A. For example, the transference mechanism 430 can move the ultrasound transducer assembly 10 along four linear segments along different sides of the container 420. For each of the segments, the ultrasound transducer assembly 10 faces inward toward the axis, A, and acquires data from a perspective different from that of the other segments. In some implementations, the path 435a can acquire data along a medial side, a lateral side, an anterior side, and posterior side of the knee 40 when the leg 44 is placed in the container 420.

Referring to FIG. 5B, the path 435b includes many segments substantially parallel to the axis, A, connected by circumferential segments about the axis, A.

Referring to FIG. 5C, the path 435c includes a helical shape extending about the axis, A. In some implementations, the path 435 has a constant radius or distance between the ultrasound transducer assembly 10 and the axis, A. When following the path 435c, the transference mechanism 430 moves the ultrasound transducer assembly 10 with a spiral motion, gradually translating the ultrasound transducer assembly 10 in a direction parallel to the axis, A, while also moving the ultrasound transducer assembly 10 circumferentially about the axis, A. Acquiring ultrasound scan data along the path 435c can provide enhanced resolution compared to other paths. The path 435c permits acquisition of data during a single, continuous motion, which can facilitate generation of a 3D surface using the scan data.

Referring to FIG. 5D, the path 435d includes round or helical segments that extend partially or completely about the axis, A, connected by segments that extend substantially parallel to the axis, A.

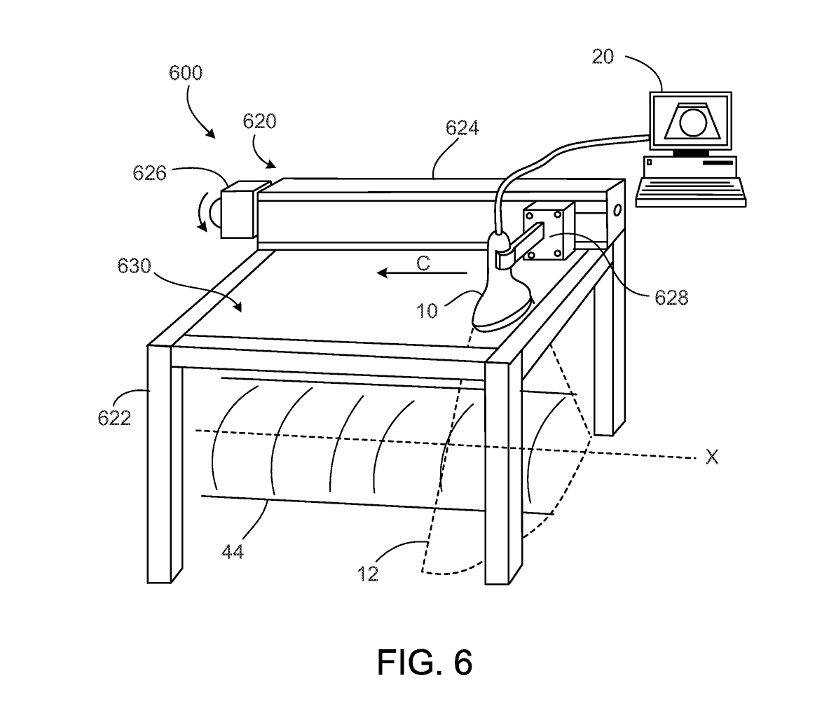

Referring to FIG. 6, a system 600 for ultrasound scanning includes the ultrasound transducer assembly 10, the control unit 20, and an automated scanning assembly 610. The automated scanning assembly 610 includes a transference assembly 620 that can be placed in a container (not shown) that holds an ultrasound-conductive medium.

The transference assembly 620 includes a frame 622 and a transference mechanism 624 mounted to the frame 622. The frame 622 provides a fixed reference position relative to which different orientations of the ultrasound transducer assembly 10 can be measured. In use, the frame 622 can be immersed in the ultrasound-conductive medium, for example, water. The frame 622 defines an opening 630 that permits the ultrasound transducer assembly 10 to be immersed in the ultrasound-conductive medium and have unimpeded access to the subject of the scan. The transference mechanism 624 attaches to the ultrasound transducer assembly 10 and moves the ultrasound transducer assembly 10 along a desired scan direction, for example, along an axis, C, that is parallel to an axis, X, of a subject, such as the leg 44. The transference mechanism 624 includes a stepper motor 626 or other element that drives motion of a moveable element 628 along, for example, a controlled linear path. The motor 626 may move the ultrasound transducer assembly 10 in a continuous motion, or in a series of discrete movements permitting the ultrasound transducer assembly 10 to remain at rest when acquiring a data set.

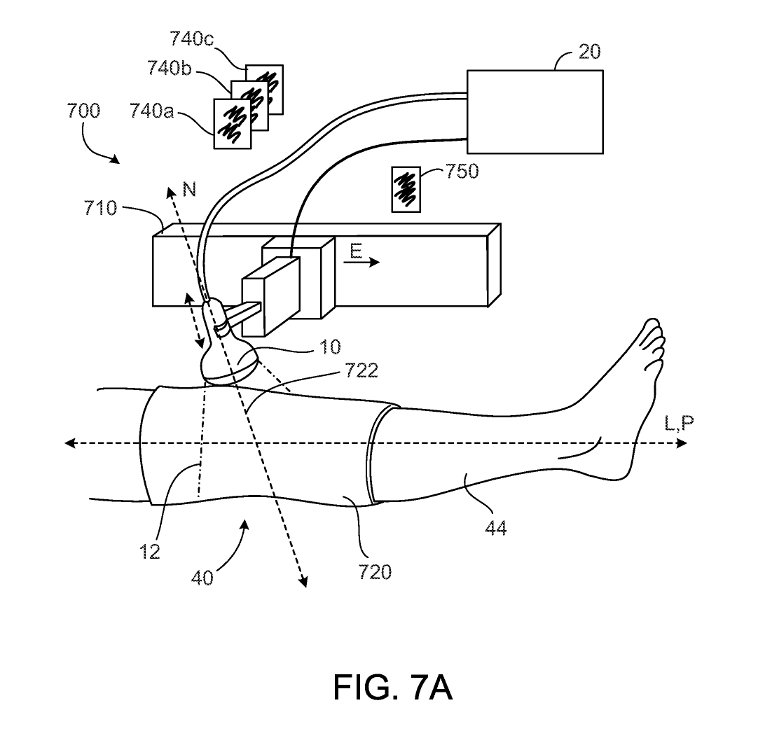

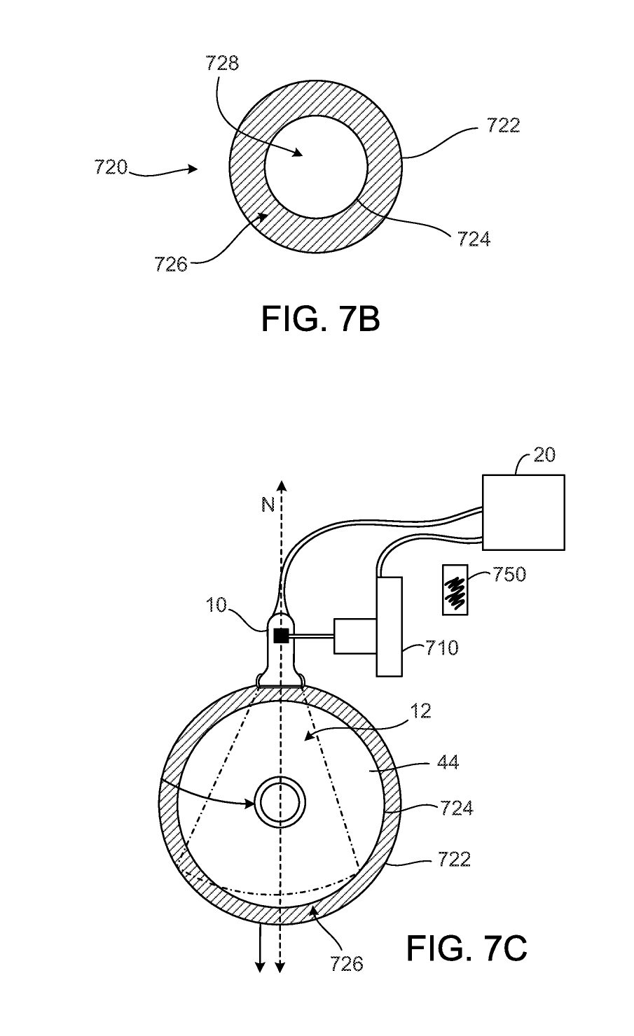

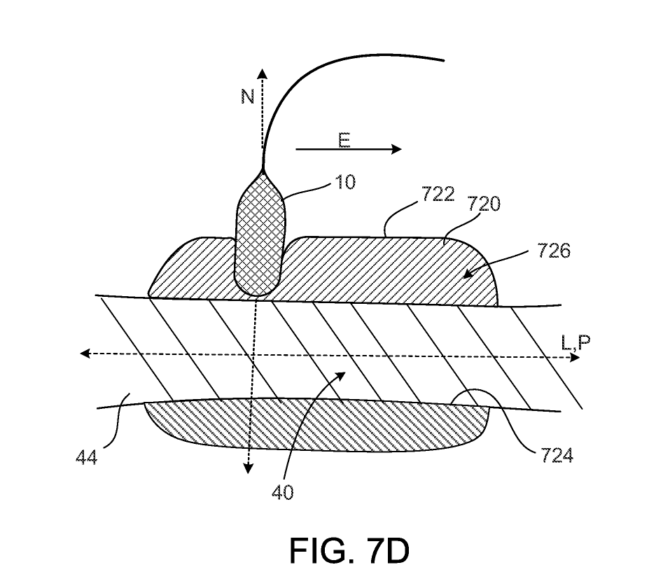

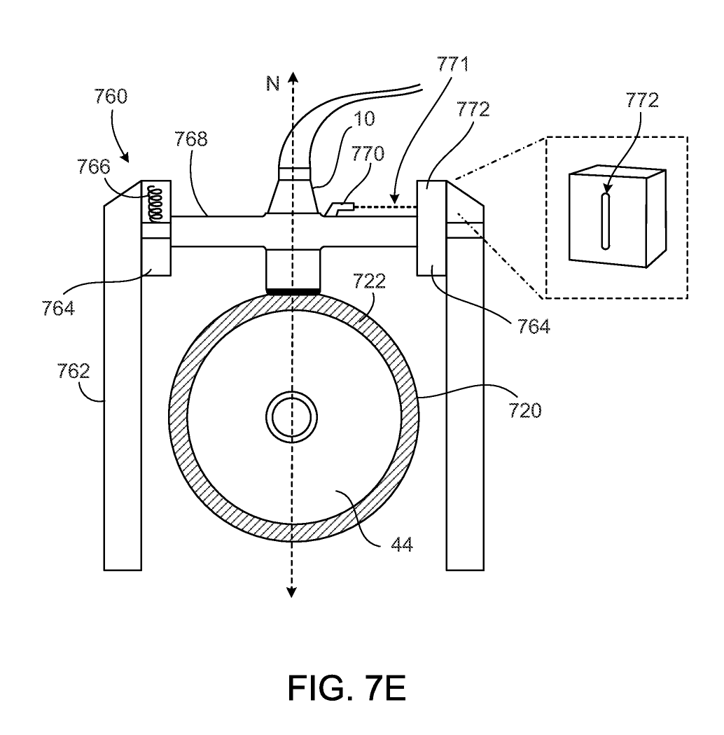

Referring to FIG. 7A, a system 700 for ultrasound scanning includes the ultrasound transducer assembly 10, the control unit 20, a transference mechanism 710, and a cuff 720. The cuff 720 extends about an anatomical region of interest, such as the knee 40, and is filled with an ultrasound-conductive medium. The transference mechanism 710 engages the ultrasound transducer assembly 10 against an outer surface 722 of the cuff 720, such that ultrasound is transmitted through the cuff 720 to bone, and reflected ultrasound is transmitted through the cuff 720 to the ultrasound transducer assembly 10. The transference mechanism 710 moves the ultrasound transducer assembly 10 relative to the knee 40 and the cuff 720, while maintaining contact between the ultrasound transducer assembly 10 and the cuff 720. The cuff 720 deforms in response to pressure from the ultrasound transducer assembly 10, permitting the ultrasound transducer assembly 10 to maintain contact with the cuff 720.

Referring to FIG. 7B, the cuff 720 can be formed of an ultrasound-conductive material. As a result, the cuff 720 transmits ultrasound when filled with an ultrasound-conductive medium. The cuff 720 defines a generally cylindrical cavity 726 between an inner surface 724 and the outer surface 722. The cavity 726 is filled with an ultrasound-conductive medium, such as water or gel. Air bubbles, which block the transmission of ultrasound, can be removed from the cavity 726 when the ultrasound-conductive medium is introduced.

The cuff 720 also defines an opening 728 that admits, for example, the leg 44. The cuff 720 defines a central axis, P, through the opening 728 that generally extends parallel to or collinear with the longitudinal axis, L, of the leg 44 when the cuff 720 is placed about the leg 44. The inner surface 724 of the cuff 720 conforms to the skin of the leg 44, such that air pockets do not form between the skin and the inner surface 724. An ultrasound-conductive gel can be applied to the knee 40 before placing the cuff 720 over the knee 40 to further displace air between the cuff 720 and the skin.

The cuff 720 can be pressurized to resist pressure of the ultrasound transducer assembly 10 and to conform to the patient's skin. The cuff 720 can include a pressurizing mechanism. As an example, a pump or other mechanism can supply an ultrasound-conductive medium into the cavity 726 to maintain a pressure within the cuff 720. As another example, a constricting element, such as a compressive band or a clamp, can squeeze a portion of the cuff 720 to maintain a desired pressure in the cuff 720.

Referring to FIG. 7C, the transference mechanism 710 applies pressure between the ultrasound transducer assembly 10 and the outer surface 722 of the cuff 720. For example, a motor or spring-loaded mechanism of the transference mechanism 710 presses the ultrasound transducer assembly 10 against the cuff 720 along an axis, N, that is substantially orthogonal to the longitudinal axis, L, of the leg 44, and the central axis, P, of the cuff 720. The transference mechanism 710 also permits the position of the ultrasound transducer assembly 10 to vary along the axis, N, to permit the ultrasound transducer assembly 10 to travel over the knee 40, or other irregular features. As a result, the distance between the ultrasound transducer assembly 10 and the longitudinal axis, L, is not constant as the ultrasound transducer assembly 10 travels relative to the cuff 720 and the leg 44.

As the transference mechanism 710 moves the ultrasound transducer assembly 10 during scanning, the transference unit 710 measures positions along the axis, N. For example, the transference mechanism 710 measures changes in position or an absolute position of a moveable element coupled to the ultrasound transducer assembly 10. The transference mechanism 710 measures the position with encoders or other measuring devices. The transference mechanism 710 transmits position data 750 indicating the position along the axis, N, to the control unit 20.

During scanning, the control unit 20 receives ultrasound scan data that includes data sets acquired at different positions of the ultrasound transducer assembly 10 relative to the cuff 720. The control unit 20 receives the position data 750, which is indicative of the different positions of the ultrasound transducer assembly 10 when the data sets were acquired. The control unit 20 associates each of the data sets with the position data 750 indicative of the position of the ultrasound transducer assembly 10 when the data set was acquired. The control unit 20 uses the position data 750 to compensate for the varying distance between the ultrasound transducer assembly 10 and the knee 40 when constructing a 3D representation of the knee 40.

Referring to FIG. 7D, the cuff 720 deforms in response to pressure of the ultrasound transducer assembly 10 against the outer surface 722. The transference mechanism 710 (not shown) moves the ultrasound transducer assembly 10 relative to the cuff 720 along a path while maintaining contact between the ultrasound transducer assembly 10 and the cuff 720. For example, the transference mechanism 710 moves the ultrasound transducer assembly in a direction, E, substantially parallel to the central axis, P, of the cuff 720 (and/or the longitudinal axis, L, of the leg 44). The transference mechanism 710 also generates position data indicative of different positions of the ultrasound transducer assembly 10 along the path. For example, as the cuff 720 deforms and moves along the axis, N, the transference mechanism 710 provides the position data 750 that indicates positions of the ultrasound transducer assembly 10 that correspond to different distances from the central axis, P, defined through the cuff 720.

The path that the ultrasound transducer assembly 10 travels can include one or more segments that include motion along a plane that includes the central axis, P, of the cuff 720. For example, the path can include motion in the plane that includes the axes N and P. The path can include motion in other planes that include the central axis, P, for example, along other sides of the cuff 720. The path can include one or more segments along a substantially helical direction about the central axis, P. In addition, the transference mechanism 710 can move the ultrasound transducer assembly 10 along paths as illustrated in FIGS. 5A to 5D.

Referring to FIG. 7E, a transference mechanism 760 includes a frame 762, one or more stepper motor rails 764 mounted to the frame 762, and a spring 766 to press the ultrasound transducer assembly 10 against the cuff 720. The frame 762 defines a fixed reference position relative to which different orientations of the ultrasound transducer assembly 10 can be measured. A moveable transducer holder 768 moves along the axis, N, relative to the frame 762. A stepper motor (not shown) moves the transducer holder 768 along the longitudinal axis, L, of the leg 44 during scanning.

The transducer holder 768 includes a light source 770 that directs light 771 toward a light sensor 772 coupled to the frame 762. Thus the light source 770 moves relative to the light sensor 772 when the transducer holder 768 moves relative to the frame 762. In some implementations, the light sensor 772 moves with the transducer holder 768 along the longitudinal axis, L, of the leg (e.g., along the stepper motor rails 764) but does not move along the axis, N. The light sensor 772 detects changes in the position of the transducer holder 768 along the axis, N, when the location that incident light strikes the light sensor 772 changes.

Using the manual scanning or the automated scanning described above, various techniques can be used to minimize interference of the patella when imaging articular surfaces of the knee 40. For example, scan data can be acquired with the knee 40 in different angles of flexion, where different portions of the articular surfaces are visible. In addition, when the knee 40 is in extension, tension on the patella is diminished. While the knee 40 is in extension, an operator can move the patella medially and acquire scan data of previously occluded articular surfaces. The operator can similarly move the patella laterally and acquire scan data of the previously occluded articular surfaces. The operator can use an instrument made of an ultrasound-conductive material to perturb the patella and maintain the displaced position during the scan. For example, an operator may insert an instrument inside the cuff 720 of FIG. 7A or into the container 420 of FIG. 4A to position the patella for scanning.

In addition, any of several ultrasound transducer assemblies can be used with the scanning techniques described above. For example, the references 210, 210b-210e and transference mechanisms 430, 620, 710, 760 can be configured to attach to multiple different transducer assemblies. Each can include a frame or fixture that can accommodate any of multiple different transducer assemblies. By attaching an ultrasound transducer assembly at a predetermined orientation, or by determining a positional offset with a registration process, the scanning systems described above can operate with commercially available transducer assemblies, including transducer assemblies that are already in use.

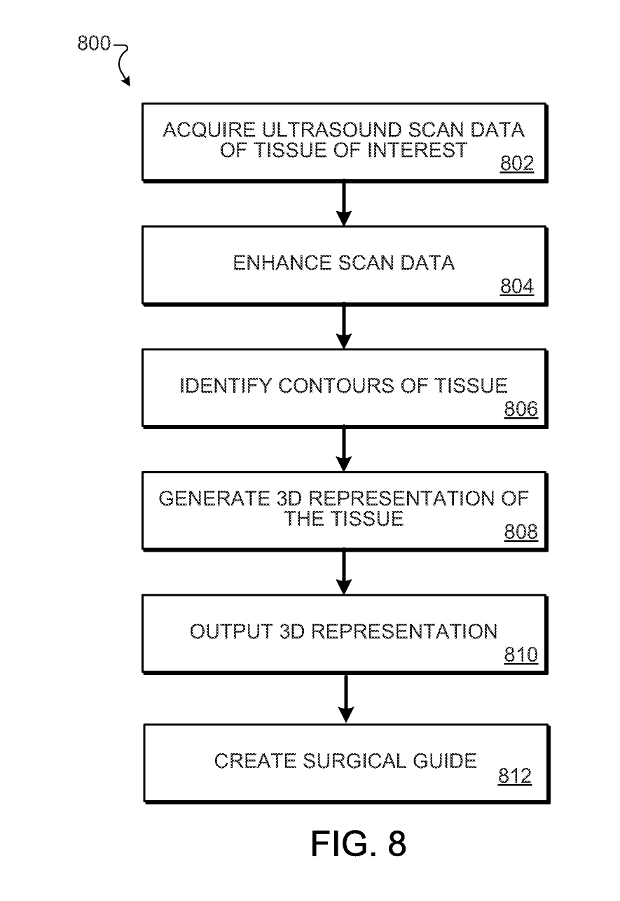

Referring to FIG. 8, a process 800 can be used to create a patient-specific surgical guide using ultrasound imaging data. Ultrasound scan data is acquired for tissue of interest (802). Ultrasound imaging data can be acquired using manual scanning or automated scanning, and with or without tracking the position of the ultrasound transducer assembly during scanning, as described above.

The scan data is enhanced (804). Noise reduction, smoothing algorithms, and filters can be applied to reduce speckle and other artifacts. Processing can be applied to individual data sets or image slices, portions of data sets, or to groups of multiple data sets. Contours of tissue are identified (806). Segmentation algorithms can be used to determine the boundaries of bone or other tissue of interest.

A 3D representation of the tissue is generated (808). The 3D representation can be a digital model that is compatible with computer aided design (CAD) software. In some implementations, techniques similar to those used to generate 3D representations from CT and MRI images are used. Some algorithms that can be used include a level set algorithm, a radial basis function, a marching cubes algorithm, and interpolation using a voxel array can be used to generate a 3D surface. When position data is associated with the imaging data sets, a processing system can use interpolation to generate data sets with regular spacing between image slices before applying the various surface modeling algorithms. When position data of the subject is collected during scanning, a processing system can also compensate for movements of the subject.

In some implementations, the system creates a 3D surface using points representing the surface of the bone. Points from multiple data sets that correspond to the surface of the bone can be registered in a common coordinate reference system, resulting in a “point cloud.” Data fitting algorithms can then be used to generate surfaces based on the characteristics of the point cloud.

The 3D representation of the tissue is output (810). For example, a CAD model can be transmitted to a manufacturing system. A surgical guide with patient-specific features is created using the 3D representation (812). For example, contours of the CAD model can be used to construct a surgical guide that conforms to the patient's anatomy. The surgical guide can be a cutting block that engages contours or features of bone, cartilage, or other tissue in a predetermined orientation. With the surgical guide engaged to the bone in the predetermined orientation, the surgical guide can indicate alignments for tissue resection or alignments for the positioning of other surgical tools. The surgical guide can be formed using, for example, 3D printing, molding, machining, or other techniques. The surgical guide can be, for example, a femoral or tibial cutting block that is used to define tissue resection alignments during knee arthroplasty surgery. Other surgical guides for arthroplasty of other joints or for other types of procedures can be formed in the same manner.

In addition to or as an alternative to using the CAD model to create a surgical guide, the CAD model may be used to select or adjust a prosthesis component, a surgical instrument, a surgical technique or approach, or another aspect of a patient's treatment. As a result, the CAD model can also be used for pre-operative research, implant design, and improvement of existing implant designs. For example, patient-specific implants can be designed and created using the CAD model.

A number of implementations and alternatives have been described. Nevertheless, it will be understood that various modifications may be made without departing from the spirit and scope of the disclosure. For example, while the illustrated examples show imaging of a knee, the same techniques can be used to image any bony anatomy, including a leg, an arm, a foot, a hand, a spine, a cranium, an elbow, a wrist, a finger, a toe, a hip, a shoulder, an ankle, and other bones and joints. Accordingly, other implementations are within the scope of the following claims.

1. A system for ultrasound scanning comprising:
an ultrasound transducer assembly configured to generate ultrasound scan data indicative of surface features of bony anatomy;
a first reference attachable to the ultrasound transducer assembly and configured to move with the ultrasound transducer assembly when attached to the ultrasound transducer assembly;
a second reference configured to be removably and extracutaneously secured to a patient;
a spatial tracking device configured to communicate with the first reference and the second reference;
one or more processors and one or more data storage devices storing instructions that, when executed by the one or more processors, cause the one or more processors to perform operations comprising:
receiving the ultrasound scan data, the ultrasound scan data comprising data sets acquired at different positions of the ultrasound transducer assembly relative to the bony anatomy from a direction substantially orthogonal to the bony anatomy;
receiving first position data from the first reference or the spatial tracking device, the first position data indicating different positions of the first reference during acquisition of the ultrasound scan data while the first reference is attached to the ultrasound transducer assembly;
receiving second position data from the second reference or the spatial tracking device, the second position data indicating different positions of the second reference during acquisition of the ultrasound scan data while the second reference is extracutaneously secured to a patient;
associating each data set of the ultrasound scan data with the position of the first reference and the position of the second reference when the data set was acquired; and
generating a three-dimensional representation of the bony anatomy based on the ultrasound scan data using the positions of the first reference and the second reference that are associated with the data sets to compensate for movement of the patient during acquisition of the ultrasound scan data.

2. The system of claim 1, further comprising a third reference configured to be removably and extracutaneously secured to a patient;
wherein the operations further comprise:
receiving third position data from the third reference or the spatial tracking device, the third position data indicating different positions of the third reference during acquisition of the ultrasound scan data while the third reference is extracutaneously secured to the patient; and
associating each data set of the ultrasound scan data with the position of the third reference when the data set was acquired;
wherein generating the three-dimensional representation of the bony anatomy based on the ultrasound scan data comprises generating the three-dimensional representation of the bony anatomy based on the ultrasound scan data using the positions of the first reference, the second reference, and the third reference that are associated with the data sets to compensate for movement of the patient during acquisition of the ultrasound scan data.

3. The system of claim 2, wherein receiving the second position data comprises receiving second position data indicating different positions of the second reference while the second reference moves with a tibia of the patient;
wherein receiving the third position data comprises receiving third position data indicating different positions of the third reference while the third reference moves with a femur of the patient;
wherein generating the three-dimensional representation of the bony anatomy comprises generating a three-dimensional representation of a knee joint comprising portions of the tibia and the femur.

4. The system of claim 3, wherein the scan data comprises data sets acquired from each of a medial side, a lateral side, a posterior side, and an anterior side of the knee.

5. The system of claim 3 wherein the scan data comprises data sets acquired while the knee is in multiple different angles of flexion.

6. The system of claim 1, wherein the ultrasound transducer assembly is configured to produce ultrasound at a frequency of between of 5 MHz to 10 MHz;
wherein each of the data sets of the scan data represents a transverse image of a portion of the bony anatomy.

7. The system of claim 1, wherein generating the representation of the bony anatomy comprises generating a three-dimensional surface using on at least one of a level set algorithm, a radial basis function, a marching cubes algorithm, or interpolation using a voxel array.

8. The system of claim 1, wherein generating the representation of the bony anatomy comprises registering at least a plurality of the data sets relative to each other based on anatomical features indicated in the data sets.

9. The system of claim 8, wherein registering at least a plurality of the data sets relative to each other based on anatomical features indicated in the data sets comprises registering the plurality of data sets relative to each other based on portions of the plurality of data sets corresponding to one or more of a bone edge, a condyle, an epicondyle, and a tibial tuberosity.

10. The system of claim 1, wherein the first reference is a fiducial, and the spatial tracking device comprises an optical detector.

11. The system of claim 1, wherein the first reference is an electromagnetic field sensor, and the spatial tracking device comprises an electromagnetic field generator.

12. The system of claim 1, wherein the first reference is a radiofrequency sensor, and the spatial tracking device comprises a radiofrequency transmitter.

13. The system of claim 1, wherein the spatial tracking device is a mechanical linkage configured to measure movement, and the first reference is an attachment element attached to the mechanical linkage.

14. The system of claim 1, further comprising one or more of a brace, a sleeve, a band, a strap, a belt, a clip, an adhesive, or an elastic configured to extracutaneously attach the second reference to the anatomy of the patient.

15. The system of claim 1, wherein generating the three-dimensional representation of the bony anatomy comprises:
identifying points that correspond to surfaces of the bony anatomy based on the scan data;
registering the identified points in a coordinate reference system to form a three-dimensional point cloud; and
determining a three-dimensional surface corresponding to the bony anatomy based on the point cloud.
